(19) United States
(12) Patent Application Publication
(10) Pub. No.: US 2015/0065907 A1
(43) Pub. Date: Mar. 5, 2015

(54) APPARATUS AND METHOD FOR SELECTIVELY COLLECTING ELECTROENCEPHALOGRAM DATA THROUGH MOTION RECOGNITION

(71) Applicant: Korea Institute of Science and Technology, Seoul (KR)

(72) Inventors: Chungki LEE, Seoul (KR); Hoyeoul PARK, Namyangju-si (KR); Jungchae KIM, Seoul (KR); Laehyun KIM, Seoul (KR); Gyu Hyun KWON, Seoul (KR); Byeongnam KIM, Jeonju-si (KR)

(73) Assignee: KOREA INSTITUTE OF SCIENCE AND TECHNOLOGY, Seoul (KR)

(21) Appl. No.: 14/474,486

(22) Filed: Sep. 2, 2014

(30) Foreign Application Priority Data
Sep. 4, 2013 (KR) 10-2013-0105841

(51) Int. Cl. A61B 5/0478; A61B 5/11

(52) U.S. Cl. CPC A61B 5/0478; A61B 5/1128
USPC 600/544

(57) ABSTRACT

Disclosed is an apparatus for selectively collecting electroencephalogram (EEG) data through motion recognition including a motion recognition unit to recognize a motion of a user by analyzing an image taken through a camera, an EEG measurement unit installed at a head part of the user to measure an EEG of the user, and a control unit to control the EEG measurement unit to measure an EEG of the user during the recognized motion of the user and to generate an EEG data set based on the measured EEG, and a method using the apparatus.

### CROSS-REFERENCE TO RELATED APPLICATION

This application claims priority to Korean Patent Application No. 10-2013-0105841, filed on Sep. 4, 2013, and all the benefits accruing therefrom under 35 U.S.C. §119, the contents of which in its entirety are herein incorporated by reference.

### BACKGROUND

1. Field

The present disclosure relates to an apparatus and method for collecting electroencephalogram (EEG) data, and more specifically, to an apparatus and method that may selectively collect EEG data by recognizing a motion of a user through a camera.

2. Description of Related Art

A brain computer interface (BCI) represents an interface technology that directly connects a human brain with a computer to control the computer through electroencephalography (EEG). An electroencephalogram (EEG), a recording of signals generated from the brain as measured from electrodes, shows a combination of electrical signals generated from numerous neurons, occurring at the surface of the brain, and may be spatiotemporally variable based on brain activity, a brain state when measured, and brain functions. An EEG has a frequency in a range of 1 to 50 Hz and an amplitude in a range of around 10 to 200 μV, and is categorized into delta, theta, alpha, beta, and gamma waves based on frequency and voltage ranges.

BCI technology may receive an EEG through a device which recognizes an EEG stimulus, analyze the EEG through a signal processing operation, and output a command through an input/output device. The BCI technology identifies changes in brain activity temporally and spatially based on the EEG, namely, spontaneous electrical activity measurable from a human scalp.

As a method for EEG measurement, an invasive form which involves a procedure for placing sensors directly on the scalp and a non-invasive form which does not involve a procedure for placing sensors on the scalp are being used. In the case of the non-invasive form, contamination by artifacts is unavoidable, resulting in information loss, and the invasive form has a grave issue with a burden of a procedure. In the case of the non-invasive form, in an attempt to minimize the influence by artifacts, filtering is performed on a measured EEG to solve the shortcoming.

Also, other than contamination by artifacts, another factor that reduces performance of BCI technology is a lack of reference EEG data. The reference EEG data is a data set of optimized status with minimized noise due to reference database when decoding in BCI technology. And, in a data set obtained by extracting a feature related to a motion from an EEG, when an EEG signal irrelevant to a target motion is applied to an algorithm, BCI performance is degraded.

FIG. 1 is a diagram illustrating a process of collecting reference data according to a related art. Referring to FIG. 1, an EEG for a motion is periodically measured at a predefined sampling frequency. However, even though a motion is absent during a fourth period, EEG data acquisition is carried out, so reference dataset has a possibility for becoming the contaminated dataset due to mixing both motion-related signal and the motion-irrelevant one.

Conventional method collecting EEG data, as shown in FIG. 1, obtains EEG data by dividing time as particular time period. Therefore, the conventional method has a limitation for estimating user's motor intention by means of reference EEG dataset, because EEG information can be inappropriate interval such as too short or too long.

### SUMMARY

An apparatus for selectively collecting electroencephalogram (EEG) data through motion recognition according to an exemplary embodiment includes a motion recognition unit to recognize a motion of a user by analyzing an image taken through a camera, an EEG measurement unit installed at a head part of the user to measure an EEG of the user, and a control unit to control the EEG measurement unit to measure an EEG of the user during the recognized motion of the user, and to generate an EEG data set based on the measured EEG.

Also, in one embodiment, in the apparatus for selectively collecting EEG data through motion recognition, the EEG measurement unit may include an analog-to-digital (A/D) converter to convert, to a digital signal, an analog signal inputted through an electrode installed at the head part, a filter unit to filter the converted digital signal to amplify a necessary signal and remove a noise, and a feature extraction unit to extract a feature for the motion of the user from the filtered signal.

Also, in one embodiment, the apparatus for selectively collecting EEG data through motion recognition may further include a database (DB), and the DB may include an EEG data set for each of a plurality of user motions, and the EEG data set may be composed of EEG data including the feature.

A method for selectively collecting EEG data through motion recognition according to an exemplary embodiment includes taking an image of a user through a camera, recognizing a motion of the user by analyzing the captured image, measuring an EEG of the user using an EEG measurer installed at a head part of the user, in which the EEG of the user is measured during the recognized motion of the user, and generating an EEG data set based on the measured EEG.

### BRIEF DESCRIPTION OF THE DRAWINGS

FIG. 1 is a diagram illustrating a process of collecting reference data according to a related art.

FIG. 2 is a block diagram illustrating an apparatus for selectively collecting electroencephalogram (EEG) data through motion recognition according to an exemplary embodiment.

FIG. 3 is a block diagram illustrating an EEG measurement unit 200 in another exemplary embodiment.

FIG. 4 is a diagram illustrating an operation of an apparatus 1000 for selectively collecting EEG data through motion recognition according to an exemplary embodiment.

FIG. 5 illustrates an EEG data set according to an exemplary embodiment and an EEG data set according to a related art being compared.

FIG. 6 is a diagram illustrating a user motion.

FIG. 7a through FIG. 7d show EEG spectrum images and pattern of movement by obtaining camera.

FIG. 8 is a diagram illustrating an operation of an apparatus 1000 for selectively collecting EEG data through motion recognition according to another exemplary embodiment.

FIG. 9 is a flowchart illustrating a method for selectively collecting EEG data through motion recognition according to still another exemplary embodiment.

### DETAILED DESCRIPTION

Embodiments described herein may take the form of entirely hardware, partially hardware and partially software, or entirely software. The term “unit”, “module”, “device” or “system” as used herein is intended to refer to a computer-related entity, either hardware, a combination of hardware and software, or software. For example, a unit, module, device or system as used herein may be, but is not limited to being, a process running on a processor, a processor, an object, an executable, a thread of execution, a program, and/or a computer. By way of illustration, both an application running on a computer and the computer may correspond to a unit, module, device or system of the present disclosure.

The embodiments are described with reference to flowcharts presented in the drawings. For concise description, the method is illustrated and described as a series of blocks, but the present disclosure is not limited to an order of the blocks, and some of the blocks may be placed with the other blocks in a different order from an order illustrated and described herein or may be concurrent with the other blocks, and a variety of different branches, flow paths, and block orders achieving a same or similar result may be implemented. Also, for implementation of the method described herein, all the blocks shown herein may not be required. Further, the method according an exemplary embodiment may be implemented in a form of a computer program for performing a series of processes, and the computer program may be recorded in a computer-readable recording medium.

Hereinafter, exemplary embodiments of the present disclosure will be described in detail with reference to the drawings.

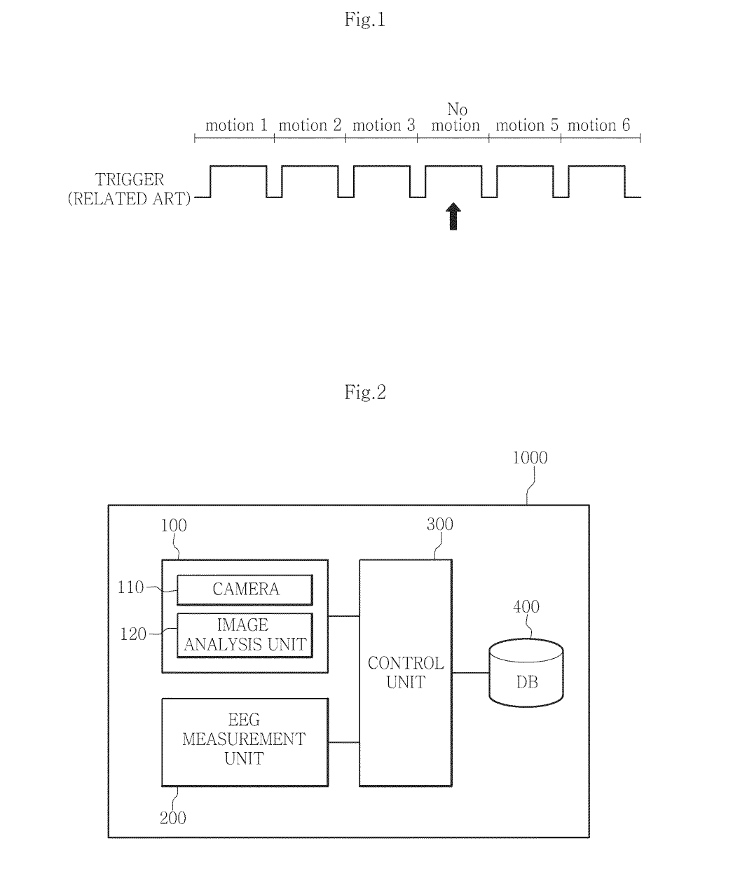

FIG. 2 is a block diagram illustrating an apparatus for selectively collecting electroencephalogram (EEG) data through motion recognition according to an exemplary embodiment of the present disclosure. In one embodiment, the apparatus 1000 for selectively collecting EEG data through motion recognition includes a motion recognition unit 100, an EEG measurement unit 200, and a control unit 300, and may further include a database (DB) 400.

In one embodiment, the motion recognition unit 100 may recognize a motion of a user by analyzing an image taken through a camera. To do so, the motion recognition unit 100 may include a camera 110 to take an image of a user, and an image analysis unit 120 to analyze a plurality of motions included in the captured image by performing image processing on the image.

To recognize the motion of the user, a three-dimensional (3D) image taken with the camera 110 is needed, and to create a 3D image, depth information of a scene is needed together with a multi-view image. A method of acquiring depth information includes a passive method and an active method, and in the present disclosure, both methods may be used. A passive method estimates depth information of a scene using a captured image and refers to a stereo matching or 2D-to-3D conversion process, and an active method is a method using a distance sensor and may use a depth camera using a time of flight (TOF) sensor and a 3D scanning machine.

The image analysis unit 120 may recognize a pose change of the user using a feature point in the captured image. For example, the image analysis unit 120 may recognize a movement of a head with respect to a defined marker on a head. In one embodiment, the image analysis unit 120 may recognize the motion of the user using any common technique for recognizing a movement of a subject from image information.

The EEG measurement unit 200 is installed at a head part of the user to measure an EEG of the user. Specifically, the EEG measurement unit 200 may perform EEG signal acquisition through electrodes attached to the head, preprocessing of the acquired EEG signal, and feature extraction. For EEG measurement, a sampling frequency, a gain, and a measurement channel may be preset.

FIG. 3 is a block diagram illustrating the EEG measurement unit 200 in another exemplary embodiment. Referring to FIG. 3, the EEG measurement unit 200 may include an analog-to-digital (A/D) converter 210, a filter unit 220, and a feature extraction unit 230. The A/D converter 210 may convert, to a digital signal, an analog signal inputted through an electrode installed at a head part. The filter unit 220 may filter the converted digital signal to amplify a necessary signal and remove a noise. As various noises are mixed in an EEG measured by a non-invasive method, it is important to filter out such noises and an ambient noise. Along with this, amplification of a necessary EEG signal maybe performed. The feature extraction unit 230 may extract a feature for the motion of the user from the filtered signal. The feature is included in EEG data and may be used to distinguish the feature of the motion taken by the user.

The image captured to recognize the motion of the user and the EEG measured from the head of the user may be temporally synchronized and inputted to the control unit 300. In another embodiment, the image and the EEG may be individually inputted to the control unit 300 and then may be temporally synchronized by the control unit 300.

The control unit 300 may be connected with the motion recognition unit 100, the EEG measurement unit 200, and the DB 400, and may exchange information with each unit 100, 200 and 400 and control each unit 100, 200 and 400.

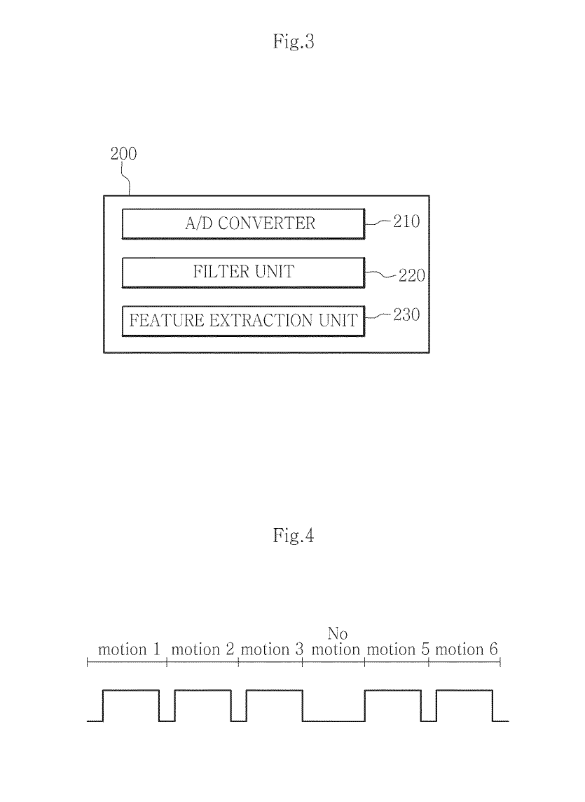

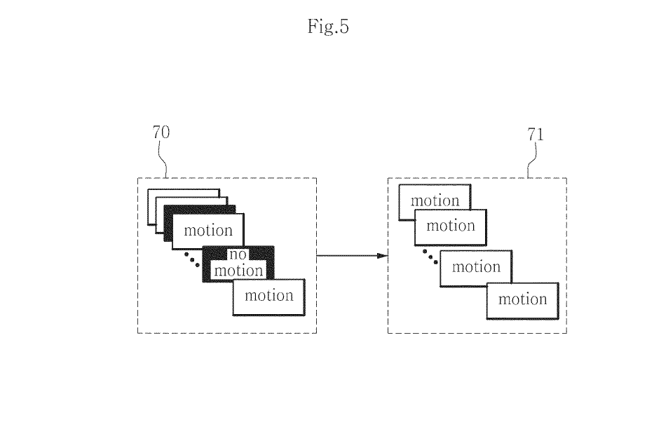

FIG. 4 is a diagram illustrating an operation of the apparatus 1000 for selectively collecting EEG data through motion recognition according to an exemplary embodiment of the present disclosure. In one embodiment, the control unit 300 may control the EEG measurement unit 200 to measure an EEG of the user during the recognized motion of the user. Referring to FIG. 4, among a total of six motion periods, a fourth motion period is a period during which a motion of the user is absent. In this case, the control unit 300 may control the EEG measurement unit 200 not to measure an EEG of the user during the fourth motion period based on information acquired from the motion recognition unit 100. Accordingly, in the case where a reference EEG data set for a particular motion is constructed based on a total of six sampling results, generation of a noise which may be included in the reference EEG data set may be prevented by excluding a motionless period.

FIG. 5 illustrates an EEG data set according to an exemplary embodiment of the present disclosure and an EEG data set according to a related art being compared. The traditional EEG data set 70 shows that EEG data is generated according to a trigger mechanism even while a motion of a user is not included. As a result, an issue with EEG data irrelevant to a particular motion being included in a data set is raised. However, the EEG data set 71 according to an exemplary embodiment of the present disclosure includes only data related to a motion of a user, and thus may have more correct data than the EEG data set 70.

In another embodiment, the apparatus 1000 for selectively collecting EEG data through motion recognition may further include the DB 400. The control unit 300 may store the generated EEG data set in the DB 400. The EEG data set included in the DB 400 may include EEG data including a feature. Here, the EEG data set may be an EEG data set for each of a plurality of user motions. For example, EEG data sets for raising a right hand and raising a left hand may be recorded in the DB 400.

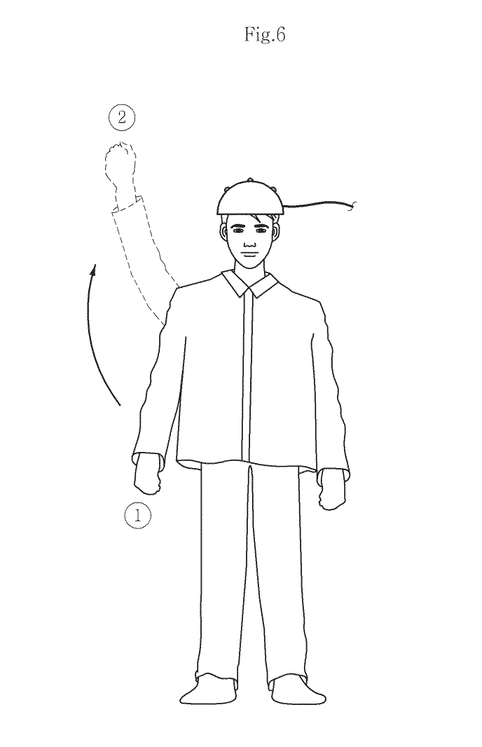

FIG. 6 is a diagram illustrating a user motion. To construct an EEG data set for a particular motion, EEG data is needed to be collected, and to collect EEG data, the foregoing-described construction may be used. Referring to FIG. 6, a description is provided taking, as an example, a case in which an EEG data set for a right hand raising motion is collected.

In FIG. 6, in the case where an arm is present at a position \textcircled{1} (a motionless case), an EEG of a user is not measured, and while the arm is moving up to a position \textcircled{2}, the control unit 300 controls the EEG measurement unit 200 to measure an EEG of a user and collect EEG data.

In the case where a user motion recognized through the motion recognition unit 100 include a plurality of motions, the control unit 300 may control the EEG measurement unit 200 to measure an EEG only during a first user motion among the plurality of motions. For convenience of description, a description is based on that a first user motion represents raising a right hand, a second user motion represents inducing noise by unnecessary movement(e.g. waggling a head, wagging a head, and trembling a body, etc.), and a third user motion represents moving a whole body from left to right or vice versa.

However, actually, because an EEG change may occur with an unnecessary movement of a finger and a movement of a whole body when a user raises a right arm, problematically, EEG data for other motion works as a noise in EEG data measured while the right arm is moving up.

To resolve this issue, the control unit 300 according to another embodiment may control the EEG measurement unit 200 to measure an EEG when a first user motion is larger than or equal to a predetermined motion range. The first user motion represents a target motion intended to be included in a data set among various motions of a user during EEG measurement and image capturing. In the case of EEG measurement for a right hand raising motion, an EEG measured while user head is moving or user expression is changing regardless of an intention of a user is a substantially unnecessary EEG data with noise. Accordingly, the control unit 300 may control the EEG measurement unit 200 to measure an EEG from a position where a right hand moves by about a few centimeters based on a captured image.

In still another embodiment, the control unit 300 may control the EEG measurement unit 200 not to measure an EEG when a second user motion among the plurality of motions taken by the user is larger than or equal to a predetermined motion range. For example, this is to prevent EEG data for inducing noise by unnecessary movement from being included when collecting EEG data for raising the right hand.

Specifically, the motion recognition unit 100 may sense and recognize a movement of a right arm and an unnecessary movement of a finger of the user. Accordingly, the control unit 300 may control the EEG measurement unit 200 not to measure an EEG while the unnecessary movements are larger than or equal to a predetermined motion range. Here, the predetermined motion range may represent above or below an arbitrary reference value set by the user or an apparatus provider.

That is, the control unit 300 may determine whether to collect an EEG or not based on magnitude of a motion for collecting EEG data and an unnecessary motion. In another embodiment, this classification may be subdivided and organized as in the following Table 1. In the following table, labeling represents classification labeling for a motion recognized for a predetermined time period.

TABLE 1

| | First user motion | Second user motion | |
|---|---|---|---|
| | (target motion | (motion not used | |
| | for EEG data | for EEG data | Determination |
| | collection) | collection) | of data type |
| Motion range | Above reference | Below reference | Data |
| | Above reference | Above reference | Data + Noise |
| | Below reference | Above reference | Noise |
| | Below reference | Below reference | Not labeling |

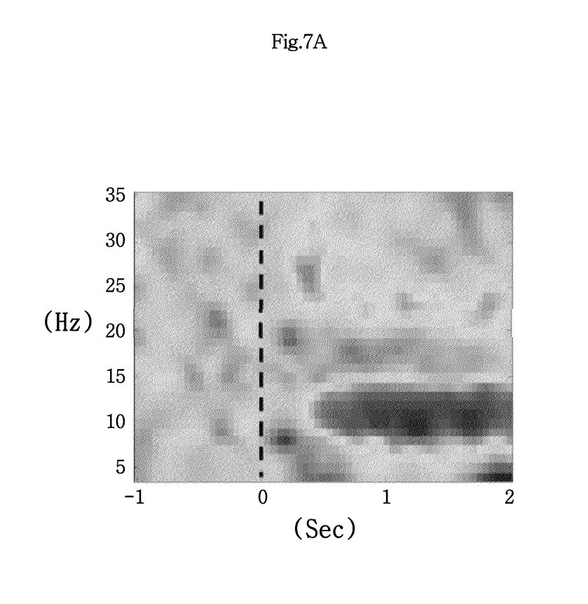

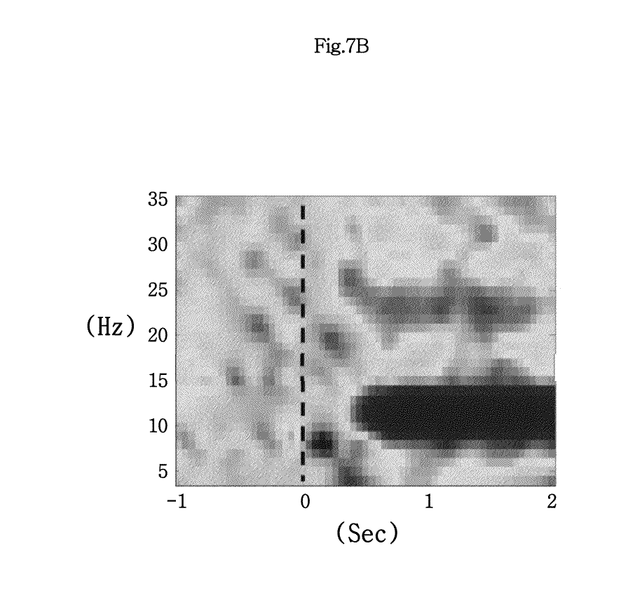

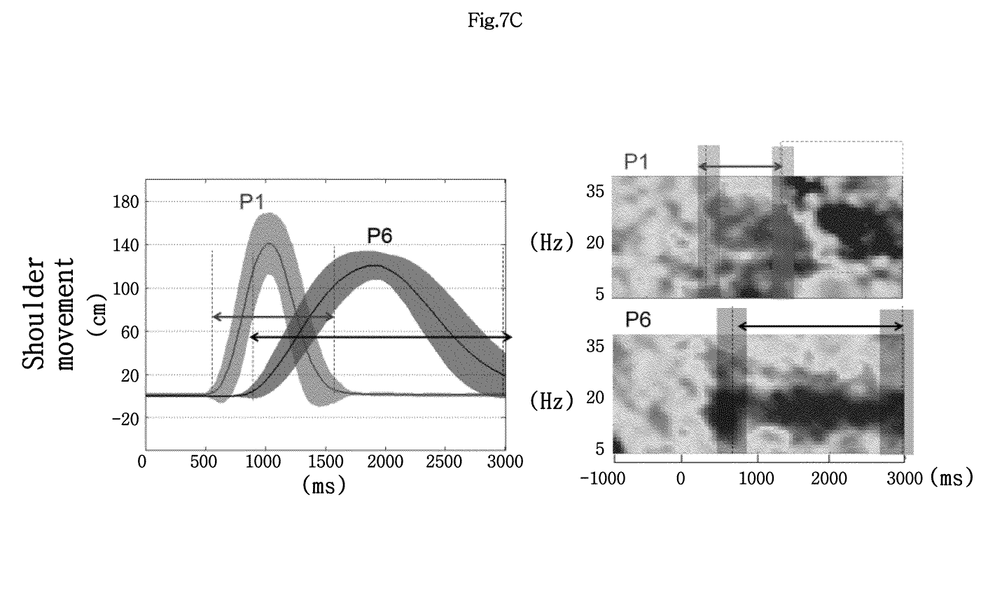

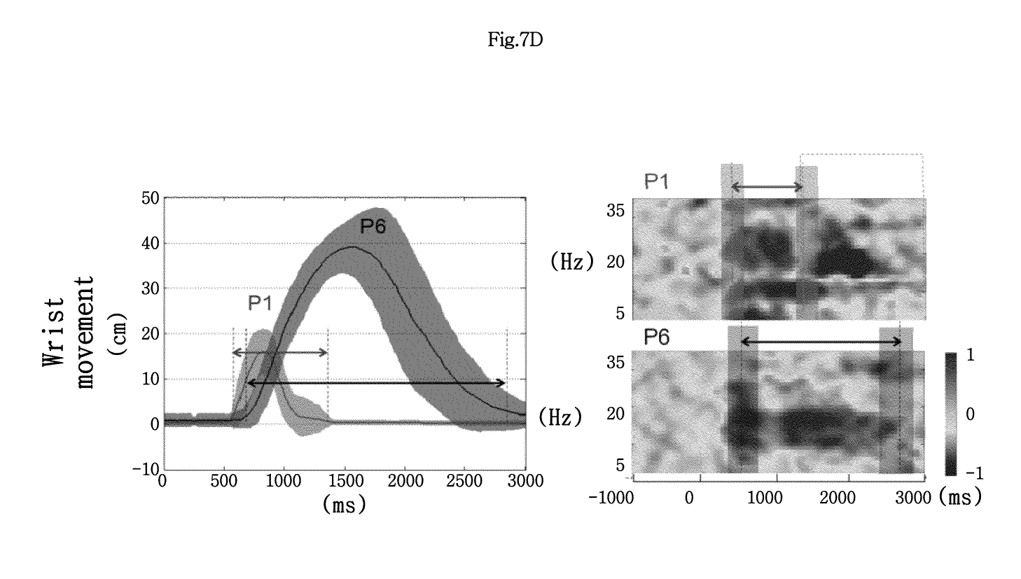

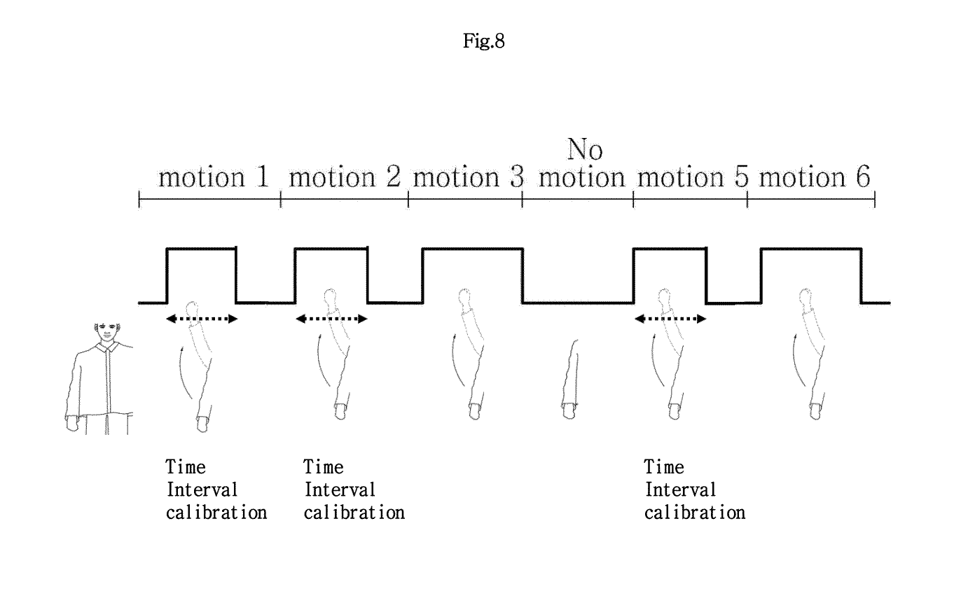

FIG. 7a through FIG. 7d show EEG spectrum images. FIG. 7a is an EEG spectrum image obtained by collecting an EEG by a periodic trigger mechanism without considering a motion of a user according to a traditional method. Also, FIG. 7b is an EEG spectrum image obtained by collecting an EEG only during a motion of a user according to an exemplary embodiment of the present disclosure. Seeing time period (1˜2 sec) after dotted circles drawn in FIGS. 7a and 7b, it is found that the EEG spectrum image obtained by collecting an EEG only during a motion of a user has higher spectrum intensity in alpha(8˜13 Hz) and beta(20˜25 Hz). Also, FIG. 7c and FIG. 7d are an EEG spectrum image with starting point calibration and ending point calibration obtained by collecting an EEG only during a motion of a user according to an exemplary embodiment of the present disclosure, in which a spectrum synchronized in movement may be obtained. Therefore, EEG signal of time domain related to necessary movement may be extracted. Here, P1 represents an user with early starting point and ending point of movement, P6 represents an user with late starting point and ending point of movement, EEG signal related to movement according to movement pattern of user may be extracted to apply different time domain for each user.) An exemplary embodiment for aforementioned method describe in FIG. 8.

FIG. 8 is a diagram illustrating an operation of an apparatus 1000 for selectively collecting EEG data through motion recognition according to another exemplary embodiment. More specifically, FIG. 8 represents an operation of an apparatus 1000 for selectively collecting EEG data through motion recognition and a method to collect simultaneously starting point and ending point of movement for operation.

The EEG data set obtained in this way may be applied to a brain computer interface (BCI) application operation. Specifically, in the case where EEG analysis is conducted to move a robot arm or a cursor on a screen, the measured EEG may be used as an element that determines what is an intention.

A method for selectively collecting EEG data through motion recognition according to an exemplary embodiment of the present disclosure may include taking an image of a user through a camera, recognizing a motion of the user by analyzing the captured image, measuring an EEG of the user using an EEG measurer installed at a head part of the user, in which the EEG of the user is measured during the recognized motion of the user, and generating an EEG data set based on the measured EEG.

In one embodiment, the measuring of the EEG of the user may include converting, to a digital signal, an analog signal inputted through an electrode installed at the head part, filtering the converted digital signal to amplify a necessary signal and remove a noise, and extracting a feature for the motion of the user from the filtered signal.

Also, a method for selectively collecting EEG data through motion recognition according another exemplary embodiment may further include storing an EEG data set for each of a plurality of user motions in a DB. Here, the EEG data set may be composed of EEG data including the feature.

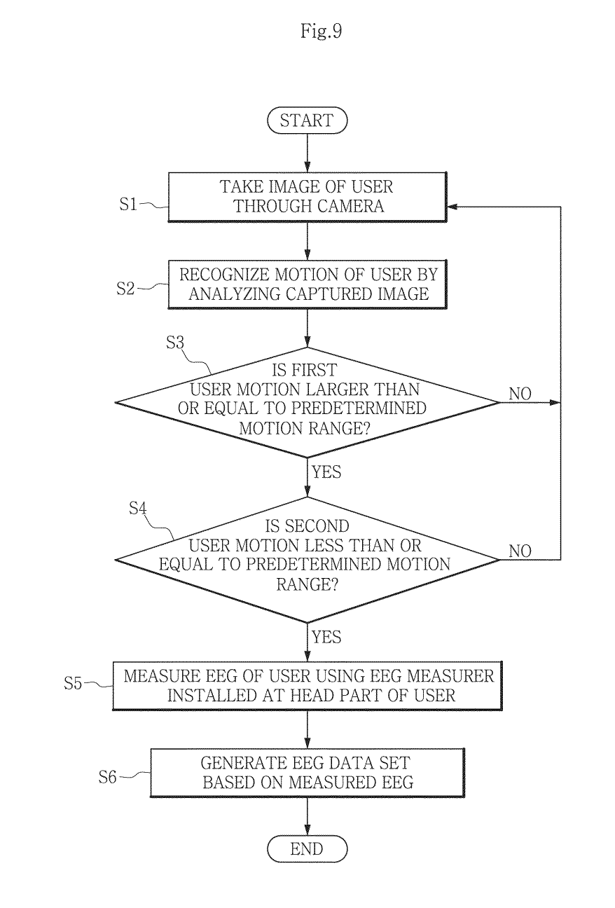

FIG. 9 is a flowchart illustrating a method for selectively collecting EEG data through motion recognition according to still another exemplary embodiment of the present disclosure. The method for selectively collecting EEG data through motion recognition takes an image of a user through a camera (S1), and recognizes a motion of the user by analyzing the captured image (S2). Along with the operations (S1) and (S2), measuring an EEG of the user may be performed through an EEG measurer installed at a head part of the user. Here, the image from the EEG measurer may be time synchronized with the image from the camera for recognition of the motion of the user.

Whether a first user motion or a target motion for EEG data collection is larger than or equal to a predetermined motion range is determined (S3), and when the first user motion is larger than or equal to the predetermined motion range, determination is made as to whether a second user motion is less than or equal to a predetermined motion range (S4). Here, the second user motion represents a motion of a different body part or having a different pattern from the first user motion.

When the first user motion is not larger than or equal to the predetermined motion range, the process reverts to S1 to take an image of the user through the camera. That is, EEG collection for a current motion is not performed. Likewise, when the second user motion is not less than or equal to the predetermined motion range, the process reverts to S1.

When the second user motion is less than or equal to the predetermined motion range, an EEG of the user is measured and collected using the EEG measurer (S5). An EEG data set is generated based on the collected EEG (S6). The generated EEG data set may be stored in a DB. Also, in an application operation using an EEG, an electrical signal corresponding to the measured EEG may be determined based on the EEG data set stored in the DB, and various applications may be executed.

While the present disclosure set forth hereinabove has been described with reference to the embodiments shown in the accompanying drawings, this is just illustrative and it will be understood by those skilled in the art that various changes in form and details may be made thereto. However, such changes should be construed as being within the technical protection scope of the present disclosure. According, the true technical protection scope of the present disclosure shall be defined by the technical doctrine of the appended claims.

What is claimed is:

1. An apparatus for selectively collecting electroencephalogram (EEG) data through motion recognition, the apparatus comprising:
a motion recognition unit configured to recognize a motion of a user by analyzing an image taken through a camera;
an EEG measurement unit configured to be installed at a head of the user for measuring an EEG of the user; and
a control unit configured to control the EEG measurement unit for measuring an EEG of the user during the recognized motion of the user, and to generate an EEG data set based on the measured EEG.

2. The apparatus according to claim 1, wherein the motion recognition unit comprises:
a camera configured to take an image of the user; and
an image analysis unit configured to analyze a plurality of motions included in the captured image by performing image processing on the image.

3. The apparatus according to claim 2, wherein the EEG measurement unit comprises:
an analog-to-digital (A/D) converter configured to convert an analog signal to a digital signal, wherein the analog signal is inputted through an electrode installed at the head;
a filter unit configured to filter the converted digital signal to amplify a necessary signal and remove a noise; and
a feature extraction unit configured to extract a feature for the motion of the user from the filtered signal.

4. The apparatus according to claim 3, wherein the apparatus further comprises a database (DB), wherein the DB includes an EEG data set for each of a plurality of user motions, wherein the EEG data set is composed of EEG data including the feature.

5. The apparatus according to claim 4, wherein the control unit causes time synchronization between the motion recognition unit and the EEG measurement unit.

6. The apparatus according to claim 5, wherein the control unit controls the EEG measurement unit to measure the EEG only during a first user motion among a plurality of motions when the recognized user motion includes the plurality of motions.

7. The apparatus according to claim 6, wherein the control unit controls the EEG measurement unit to measure the EEG when the first user motion is larger than or equal to a predetermined motion range.

8. The apparatus according to claim 7, wherein the control unit controls the EEG measurement unit not to measure the EEG when a second user motion among the plurality of motions is larger than or equal to a predetermined motion range,
wherein the second user motion is using a different body part or have a different pattern from the first user motion.

9. A method for selectively collecting electroencephalogram (EEG) data through motion recognition, the method comprising:
taking an image of a user through a camera;
recognizing a motion of the user by analyzing the captured image;
measuring an EEG of the user using an EEG measurer installed at a head of the user, wherein the EEG of the user is measured during the recognized motion of the user; and
generating an EEG data set based on the measured EEG.

10. The method according to claim 9, wherein the measuring of the EEG of the user comprises:
converting an analog signal to a digital signal, wherein the analog signal is inputted through an electrode installed at the head;
filtering the converted digital signal to amplify a necessary signal and remove a noise; and
extracting a feature for the motion of the user from the filtered signal.

11. The method according to claim 10, wherein the method further comprises storing an EEG data set for each of a plurality of user motions in a database (DB), wherein the EEG data set is composed of EEG data including the feature.

12. The method according to claim 11, the method further comprises storing an EEG data set for user motion in a database (DB), wherein the EEG data set is composed of EEG data including motion time for motion, wherein time period of collected EEG is variant.

13. The method according to claim 11, wherein the recognizing of the motion of the user comprises determining a first user motion among a plurality of motions when the recognized user motion includes the plurality of motions, wherein the measuring of the EEG of the user comprises measuring the EEG only during the first user motion.

14. The method according to claim 12, wherein the measuring of the EEG of the user comprises measuring the EEG when the first user motion is larger than or equal to a predetermined motion range.

15. The method according to claim 13, wherein the recognizing of the motion of the user comprises determining a second user motion among the plurality of motions,
wherein the measuring of the EEG of the user not to measure the EEG when the second user motion is larger than or equal to a predetermined motion range, and
wherein the second user motion is using a different body part or have a different pattern from the first user motion.
